(19) United States
(12) Patent Application Publication
(10) Pub. No.: US 2022/0126288 A1
(43) Pub. Date: Apr. 28, 2022

(54) SALIVA COLLECTION AND TRANSPORT DEVICES, SYSTEMS AND METHODS

(71) Applicant: Merit Medical Systems, Inc., South Jordan, UT (US)

(72) Inventors: Fred Lampropoulos, Salt Lake City, UT (US); Richard P. Jenkins, Bluffdale, UT (US); Gregory R. McArthur, Bluffdale, UT (US); Michael Dean Haslam, Sandy, UT (US); Kenneth Sykes, Bluffdale, UT (US); Nicole Willardson, Lehi, UT (US); Jim Mottola, West Jordan, UT (US)

(21) Appl. No.: 17/507,565

(22) Filed: Oct. 21, 2021

Related U.S. Application Data
(60) Provisional application No. 63/104,294, filed on Oct. 22, 2020.

(51) Int. Cl. B01L 3/00

(52) U.S. Cl. CPC B01L 3/50825; B01L 2200/18; B01L 2300/0832; B01L 2300/042; B01L 2200/0689

(57) ABSTRACT

Devices and methods used collect and transport a biological specimen are disclosed. The devices include a vial defining a specimen chamber to retain a specimen, a collection member to collect a specimen, and a cap containing a liquid medium to treat the specimen for transport. The cap includes a puncture member to puncture a seal to release the liquid medium from the cap.

### RELATED APPLICATION

This application claims priority to U.S. Provisional Application No. 63/104,294, filed on Oct. 22, 2020 and titled “Saliva Collection And Transport Devices, Systems And Methods” which is hereby incorporated by reference in its entirety.

### TECHNICAL FIELD

The present disclosure relates generally to devices to medical devices. More specifically, the present disclosure relates to devices used to collect and transport a biological specimen. More specifically, the present disclosure related to devices used to collect and transport a saliva specimen.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1A is a perspective view of an embodiment of a specimen device.

FIG. 1B is a cross-sectional view of the specimen device of FIG. 1A.

FIG. 2A is an exploded view of another embodiment of a specimen device.

FIG. 2B is a bottom perspective view of an embodiment of a cap of the specimen device of FIG. 2A.

FIG. 2C is a top perspective view of the cap of the specimen device of FIG. 2A.

FIG. 2D is a bottom perspective view another embodiment of the cap of the specimen device of FIG. 2A.

FIG. 2E is a cross-sectional view of a portion of the specimen device of FIG. 2A.

FIG. 2F is a cross-sectional view of the portion of the specimen device of FIG. 2A in an actuated state.

FIG. 3A is a perspective view of an embodiment of a cap having an increased height.

FIG. 3B is a cross-sectional perspective view of the cap of FIG. 3A.

FIG. 4 is a perspective view of an embodiment of a cap having a safety ring.

FIG. 5A is a cross-sectional view of another embodiment of a specimen device.

FIG. 5B is a perspective view of an embodiment of a plunger of the specimen device of FIG. 5A.

FIG. 5C is a cross-sectional view of an embodiment of a cap and the plunger of the specimen device of FIG. 5A.

FIG. 5D is a cross-sectional view of another embodiment of a cap and a plunger of the specimen device of FIG. 5A.

FIG. 6A is a perspective view of another embodiment of a specimen device.

FIG. 6B is a cross-sectional view of portion of the specimen device of FIG. 6A.

FIG. 6C is a perspective view of an embodiment of a plunger of the specimen device of FIG. 6A.

FIG. 7A is a perspective view of another embodiment of a specimen device.

FIG. 7B is a perspective view of an embodiment of a tether of the specimen device of FIG. 7A.

FIG. 7C is a perspective view of an embodiment of a collection member of the specimen device of FIG. 7A.

FIG. 8A is a perspective view of another embodiment of a specimen device.

FIG. 8B is a bottom perspective view of an embodiment of a cap of the specimen device of FIG. 8A.

FIG. 9A is a perspective view of another embodiment of a cap.

FIG. 9B is a bottom view of an embodiment of a seal member of the cap of FIG. 9A in a closed state.

FIG. 9C is a bottom view of the seal member of the cap of FIG. 9A in an open state.

FIG. 10 is a cross-sectional view of another embodiment of a specimen device with a spike insert.

FIG. 11 is a perspective view of another embodiment of a specimen device with a vial stand.

FIG. 12A is a perspective view of another embodiment of a specimen device.

FIG. 12B is a perspective view of an embodiment of a collection member of the specimen device of FIG. 12A.

FIG. 12C is a perspective view of another embodiment of a collection member of the specimen device of FIG. 12A.

FIG. 12D is a perspective view of another embodiment of a collection member of the specimen device of FIG. 12A.

FIG. 13A is a view of another embodiment of a specimen device having a medium cartridge.

FIG. 13B is a cross-sectional view of the specimen device of FIG. 13A.

FIG. 13C is a view of the specimen device of FIG. 13A in an actuated state.

FIG. 13D is a view of the specimen device of FIG. 13A with the medium cartridge removed for sampling.

FIG. 13E is a view of the specimen device of FIG. 13A with a sampling device inserted through a stopper.

### DETAILED DESCRIPTION

In certain instances, a biological specimen from a patient is collected and transported to a laboratory for analysis. In some instances, the biological specimen is saliva. The biological specimen can be analyzed to determine whether a patient is infected with a contagion caused by a virus or bacterium. In some embodiments, the virus is the SARS-CoV-2 novel coronavirus that causes Coronavirus disease 2019 (COVID-19). If the biological specimen is determined to be positive for the virus or bacterium, the patient can be treated to mitigate symptoms caused by the contagion. Further, the patient can be isolated or quarantined to prevent the spread of the contagion to non-infected people.

Embodiments herein describe specimen devices and methods used to collect and transport a fluid specimen, such as saliva, sputum, blood, semen, fetal fibronectin, amniotic fluid, cerebral spinal fluid, synovial fluid, pleural fluid, digestive secretions, urine, and other types of fluid specimens. In some embodiments within the scope of this disclosure, the specimen devices include a vial defining a specimen chamber, a collection member configured to collect the specimen, and a cap containing a liquid medium within a medium chamber. In another embodiment within the scope of this disclosure, the liquid medium is contained within the vial. In other embodiments within the scope of this disclosure, the liquid medium is contained within a medium cartridge. The liquid medium can mix with the specimen to preserve, stabilize, or react with the specimen for transport or for temporary storage.

In use, in some embodiments within the scope of this disclosure, a biological specimen is collected within the vial and an actuator of the cap is actuated to rupture or puncture a seal member with a protrusion or plunger of the cap to allow the liquid medium to flow from the cap into the vial. In another embodiment, the seal member is ruptured when the cap is coupled to a collection member and the seal member is displaced into a protrusion of the cap. In yet another embodiment, an actuator is actuated to displace a plunger within a cartridge to rupture the seal member to allow the liquid medium to flow from the cartridge into the vial.

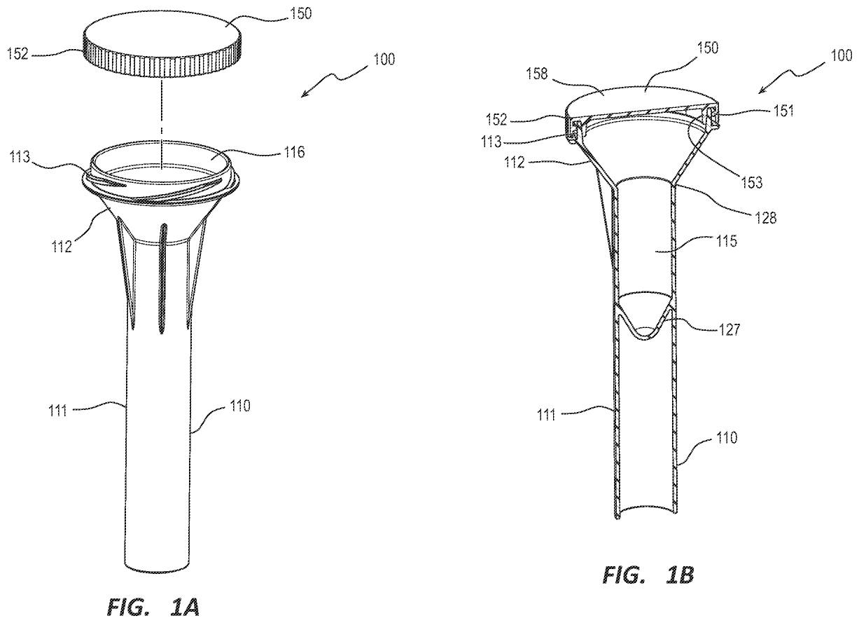

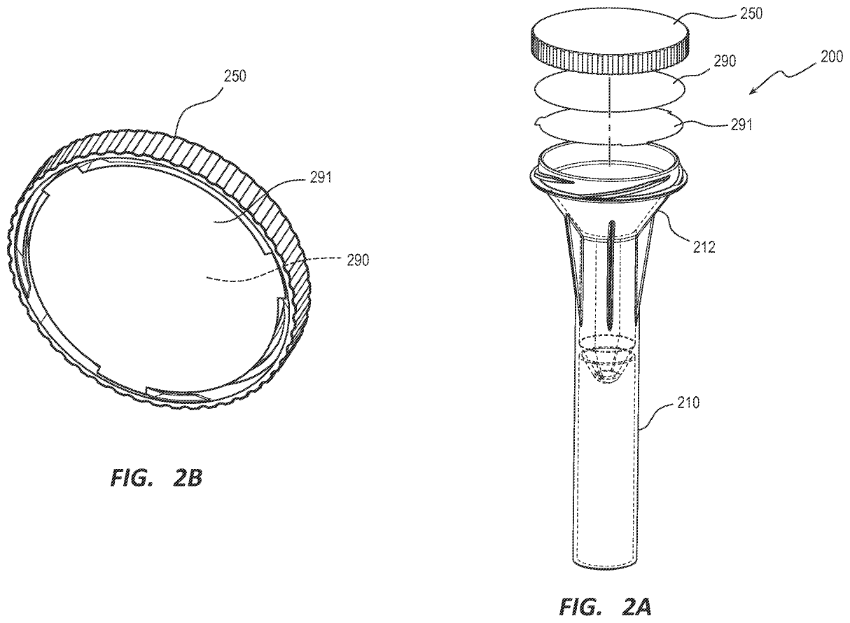

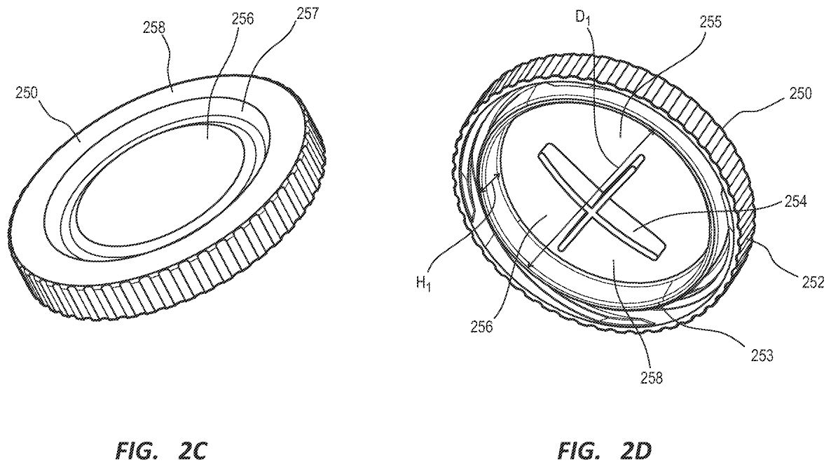

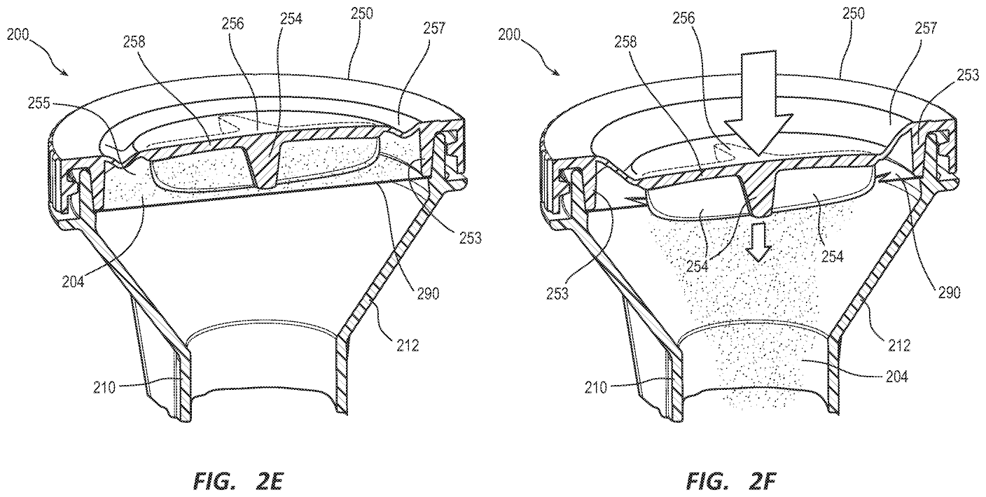

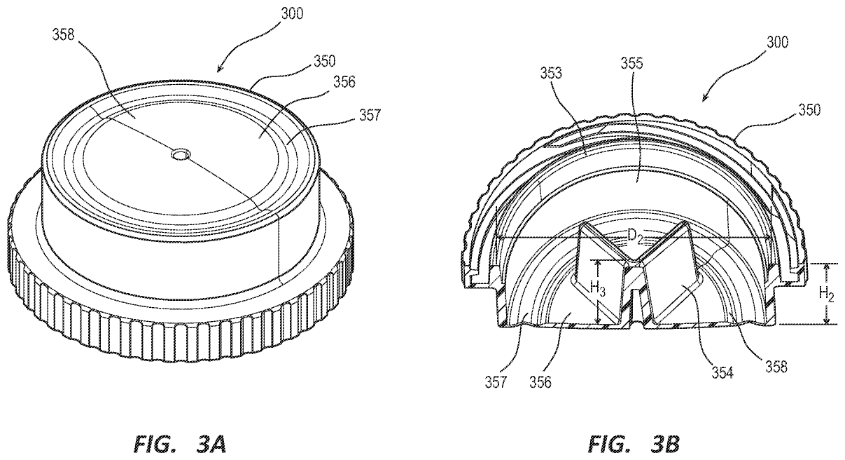

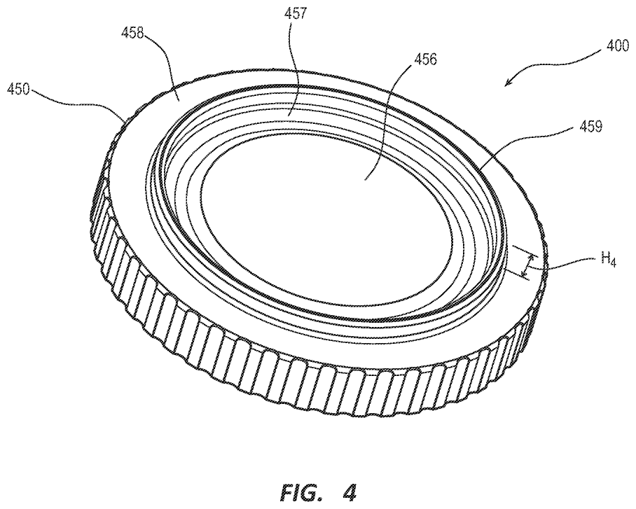

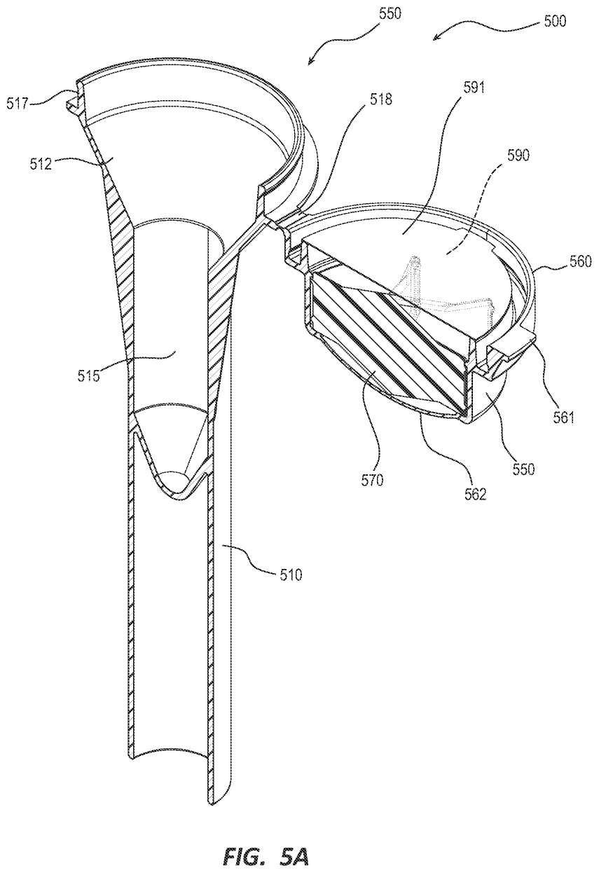

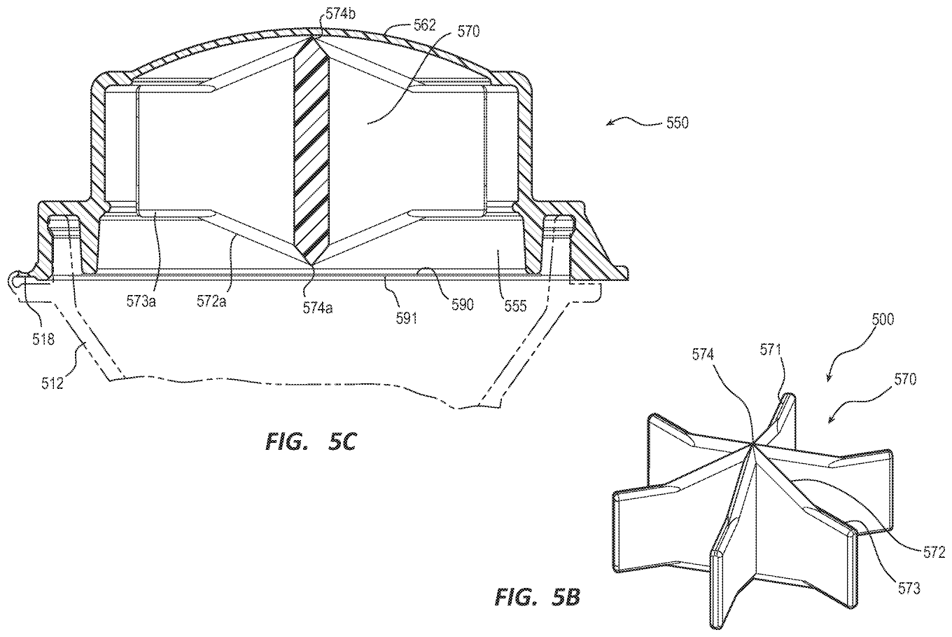

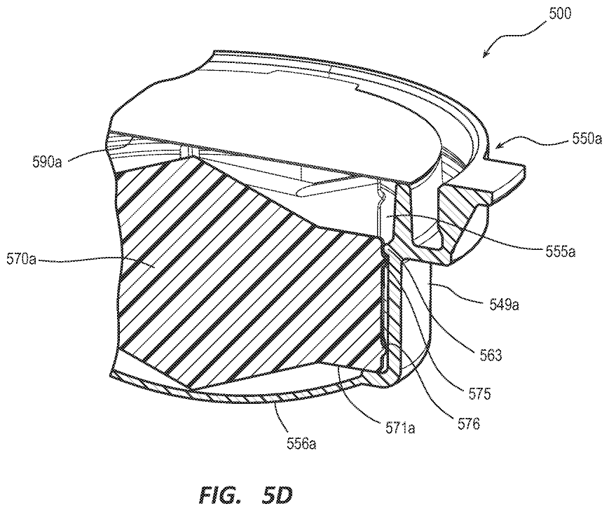

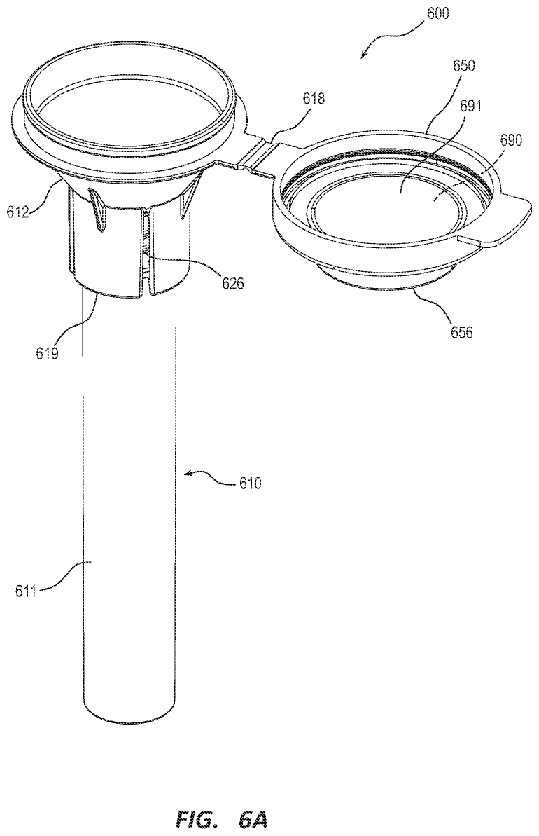

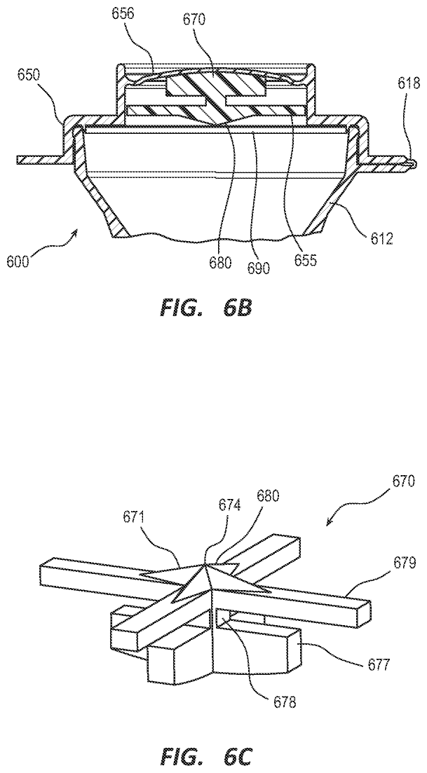

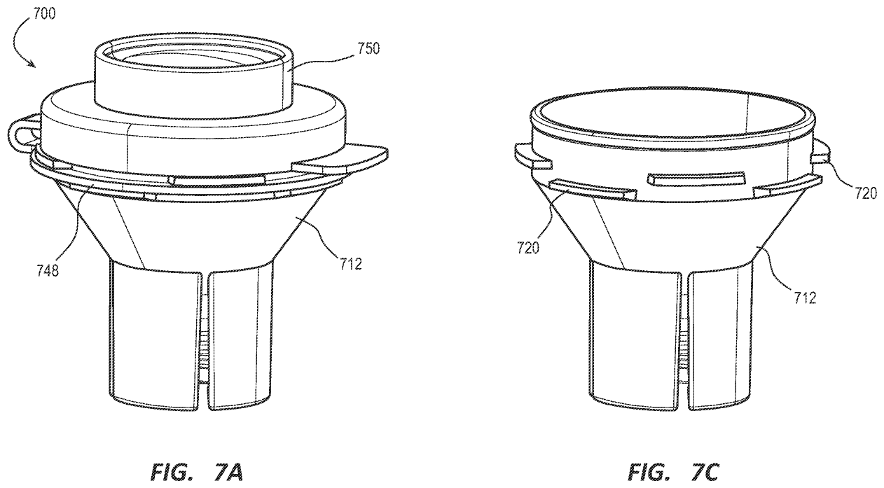

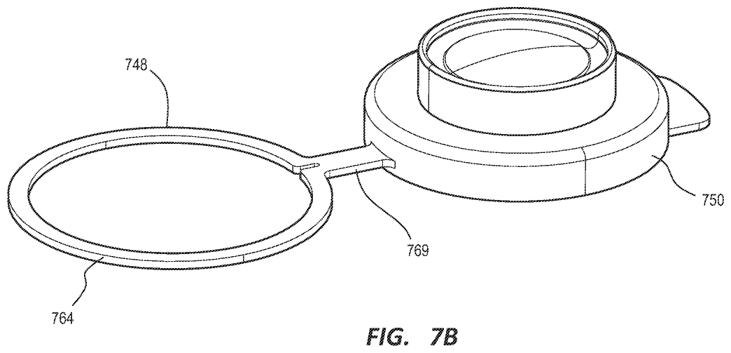

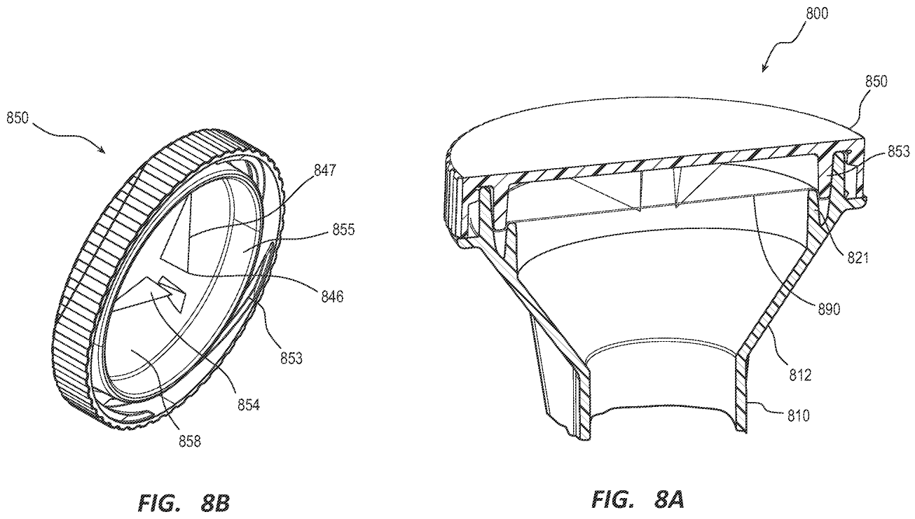

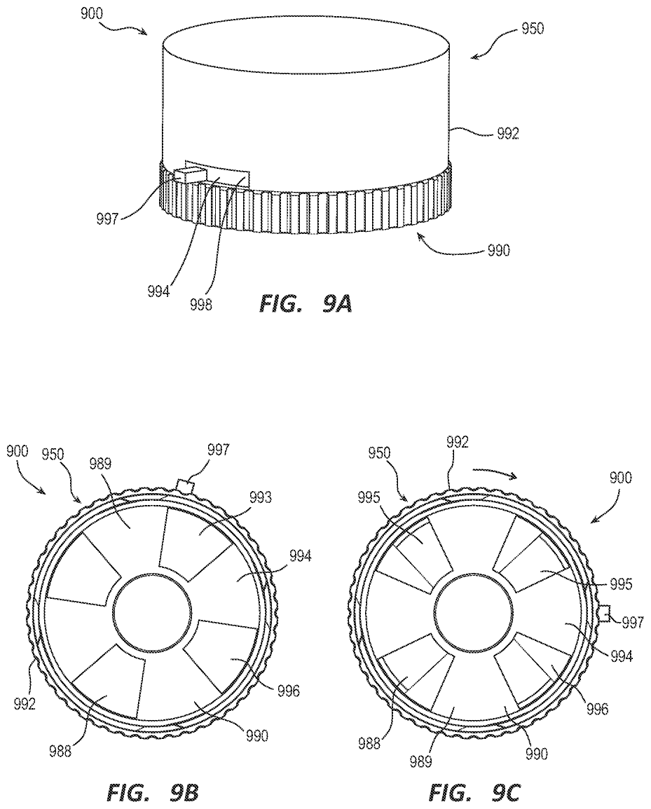

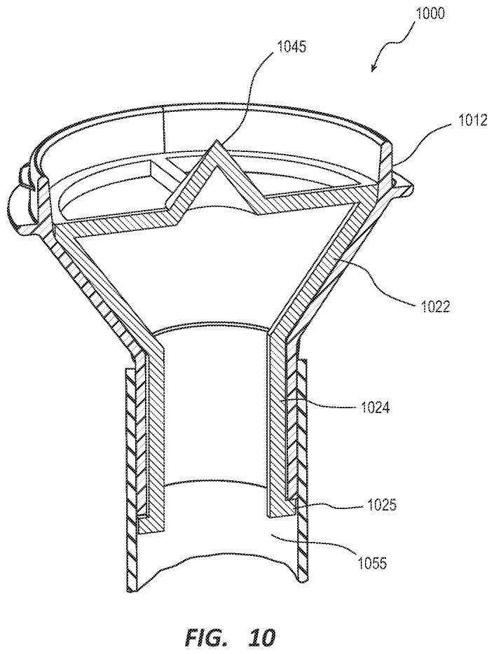

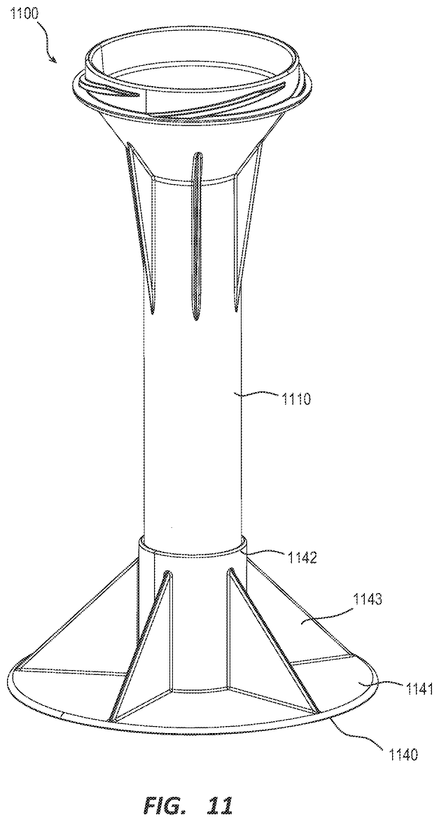

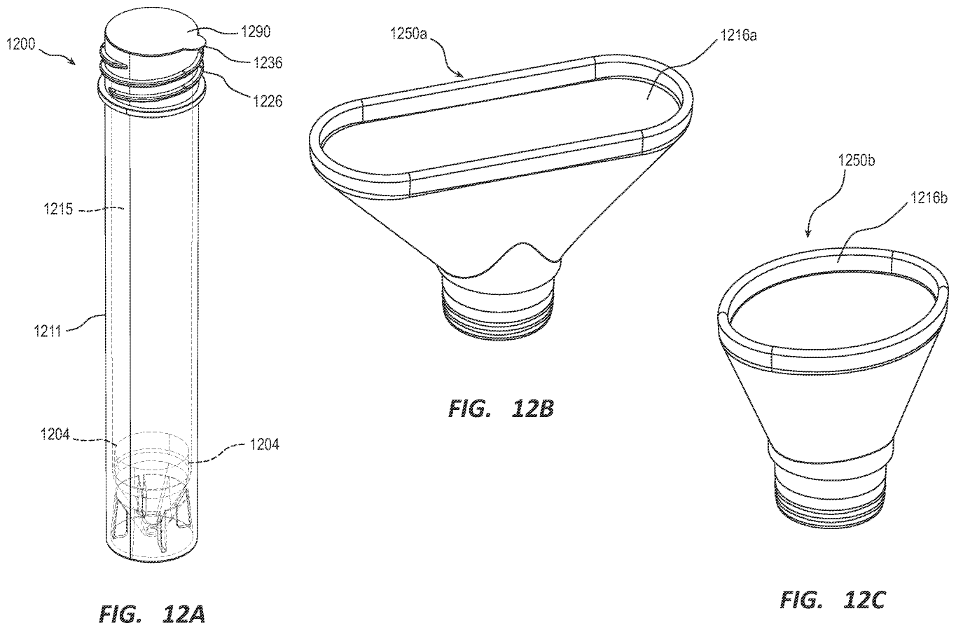

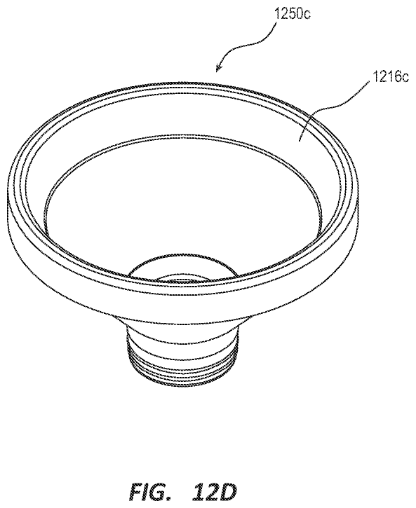

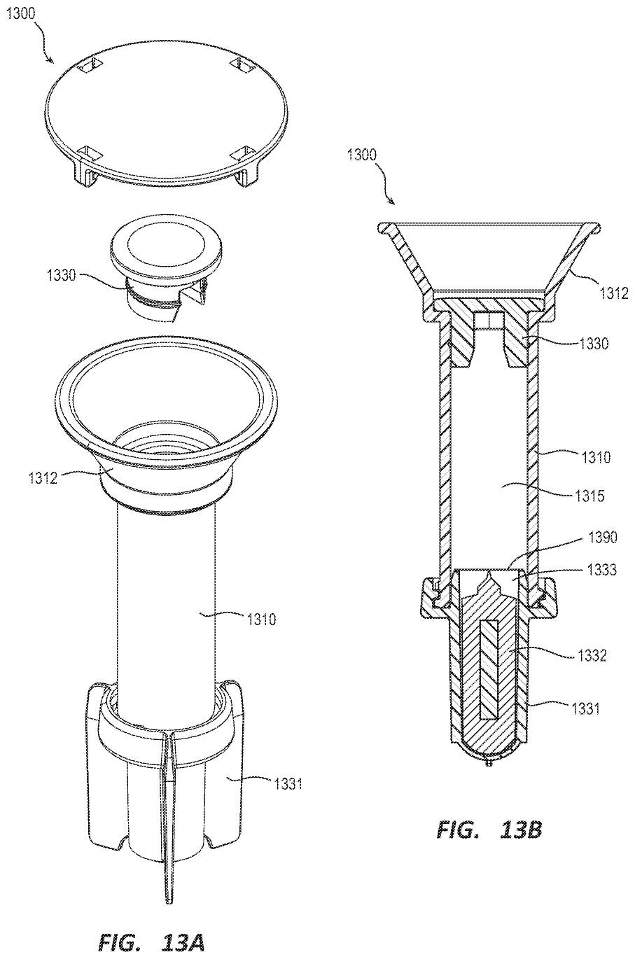

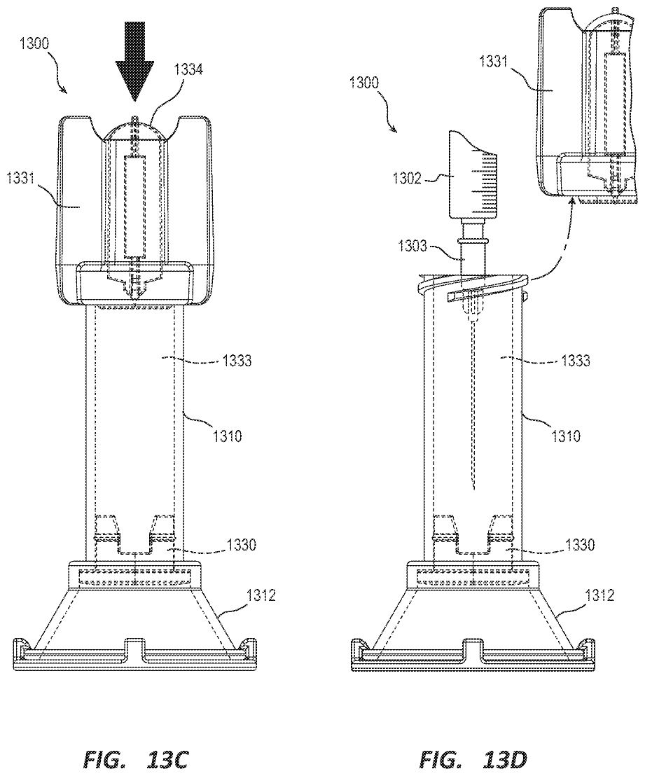

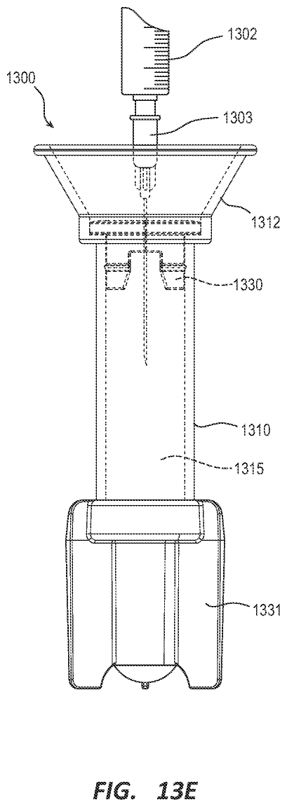

FIGS. 1A and 1B illustrate an embodiment of a specimen device including a vial, a collection member, and a cap. FIGS. 2A-2F illustrate another embodiment of a specimen device including a cap configured to contain and dispense a liquid medium. FIGS. 3A and 3B illustrate an embodiment of a cap configured to contain and dispense a larger volume of liquid medium than the cap of FIGS. 2A-2F. FIG. 4 illustrates an embodiment of a cap including a safety ring. FIGS. 5A-5D illustrate another embodiment of a specimen device including a cap including an embodiment of a plunger. FIGS. 6A-6C illustrate another embodiment of a specimen device including a cap including another embodiment of a plunger. FIGS. 7A-7C illustrate an embodiment of a collection member and a cap coupled with a tether. FIGS. 8A and 8B illustrate another embodiment of a specimen device including a collection member configured to displace a seal member into a protrusion of a cap. FIGS. 9A-9C illustrate an embodiment of a cap including a sealing member including shutter inserts. FIG. 10 illustrates another embodiment of a specimen device including a spike insert disposed within a collection member. FIG. 11 illustrates another embodiment of a specimen device including a vial stand. FIGS. 12A-12D illustrate another embodiment of a specimen device including a vial containing a liquid medium and a collector member. FIGS. 13A-13E illustrate another embodiment of a specimen device including a medium cartridge. In certain views each device may be coupled to, or shown with, additional components not included in every view. Further, in some views only selected components are illustrated, to provide detail into the relationship of the components. Some components may be shown in multiple views, but not discussed in connection with every view. Disclosure provided in connection with any figure is relevant and applicable to disclosure provided in connection with any other figure or embodiment.

As illustrated in FIG. 1A, a specimen device 100 can include three broad groups of components; each group may have numerous subcomponents and parts. The three broad component groups are: a vial 110, a collection member 112, and a cap 150.

The vial 110 may include, as depicted in FIGS. 1A and 1B, an elongate tubular body 111. A specimen chamber 115 can be defined by the tubular body 111 and may be configured to receive a biological specimen from the collection member 112 and contain the biological specimen for transport. In a certain embodiment the biological specimen is saliva. In other embodiments, the biological specimen may be any liquid secretion from a body, such as sputum, blood, semen, fetal fibronectin, amniotic fluid, cerebral spinal fluid, synovial fluid, pleural fluid, digestive secretions, urine, and other types of specimens. In other embodiments, the biological specimen may be viral transport medium, molecular transport medium, or universal transport medium. Other biological specimens are contemplated. Additionally, the specimen chamber 115 can be configured to contain a liquid medium configured to mix with and treat the biological specimen. In some embodiments the liquid medium can include any one of a preservative, an antibiotic, a pH indicator, a buffering agent, a reactant chemical, a stabilization chemical, a fixative, or any combination thereof. Other agents are within the scope of this disclosure. The specimen chamber 115 has a closed distal end 127 and an open proximal end 128. The closed distal end 127 can be disposed anywhere along a length of the tubular body 111 dependent upon a desired volume of the specimen chamber 115. For example, as depicted in FIG. 1B, the closed distal end 127 is positioned approximately at a midpoint of the tubular body 111. A volume of the specimen chamber 115 can range from about 1 milliliter to about 10 milliliters. The tubular body 111 can be formed of any suitable material, such as glass, polycarbonate, polypropylene, polyethylene, cyclic-olefin-polymer (COP), cyclic-olefin-copolymer (COC), amorphous nylon, copolyester, acetal, polyetherimide, polyetheretherketone, acrylonitrile butadiene styrene, and styrene. Other materials are contemplated within the scope of this disclosure.

As illustrated in FIGS. 1A and 1B, the collection member 112 may be coupled to the tubular body 111 and can be disposed adjacent the open proximal end 128 of the specimen chamber 115. In the depicted embodiment, the collection member 112 is integrated with the tubular body 111 as a unibody construct. In other embodiments, the collection member 112 can be a separate component and be selectively coupled to the tubular body 111. The collection member 112 may have a funnel shape that tapers inwardly from a wide opening 116 to the open proximal end 128. The wide opening 116 and the funnel shape may facilitate collecting a biological specimen from the mouth or other orifice of the patient. The collection member 112 can be in fluid communication with the specimen chamber 115 such that the collected biological specimen can flow from the collection member 112 into the specimen chamber 115. The collection member 112 may have external threads 113 configured to threadingly engage with the cap 150.

The cap 150 can be configured to fluidly seal the collection member 112 and the specimen chamber 115 to prevent spillage or contamination of the biological specimen and/or liquid medium contained within the specimen chamber 115. The cap 150 may include internal threads 151 configured to threadingly engage with the external threads 113 of the collection member 112. A sealing flange 153 can extend from a top wall 158 of the cap 150. The sealing flange 153 can sealingly couple with an internal surface of the collection member 112 as the cap 150 is coupled to the collection member 112. The cap 150 may include gripping features 152 disposed about a perimeter of the cap 150. The gripping features 152 can include knurls, bumps, divots, texturing, etc.

FIGS. 2A-2F depict an embodiment of a specimen device 200 that resembles the specimen device 100 described above in certain respects. Accordingly, like features are designated with like reference numerals, with the leading digit incremented to “2.” For example, the embodiment depicted in FIGS. 2A-2F includes a cap 150 that may, in some respects, resemble the cap 150 of FIG. 1A. Relevant disclosure set forth above regarding similarly identified features thus may not be repeated hereafter. Moreover, specific features of the specimen device 100 and related components shown in FIGS. 1A and 1B may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the specimen device 200 and related components depicted in FIGS. 2A-2F. Any suitable combination of the features, and variations of the same, described with respect to the specimen device 100 and related components illustrated in FIGS. 1A and 1B can be employed with the specimen device 200 and related components of FIGS. 1A and 1B, and vice versa. This pattern of disclosure applies equally to further embodiments depicted in subsequent figures and described hereafter, wherein the leading digits may be further incremented.

FIGS. 2A-2F illustrated another embodiment of a specimen device 200. As illustrated in FIG. 2A, the specimen device 200 may include a vial 210, a collection member 212, and a cap 250. The cap 250 can include a seal member 290 and a reinforcement member 291. FIG. 2B depicts the seal member 290 coupled to the cap 250 and the reinforcement member 291 selectively coupled to the seal member 290. The seal member 290 can include a metal foil or laminated metal foil configured to prevent evaporation of a liquid medium contained within the cap 250 and to be ruptured when the cap 250 is actuated. The reinforcement member 291 can be configured to prevent the seal member 290 from being inadvertently ruptured. The reinforcement member 291 may include a polymeric or paper material coated with a pressure-sensitive adhesive to facilitate selective coupling to and removal from the seal member 290.

FIG. 2C illustrates a top view of the cap 250 including a top wall 258. As illustrated, the top wall 258 may include a flex ring 257 and an actuator 256 circumferentially surrounded by the flex ring 257. The flex ring 257 can be configured to facilitate deflection of the actuator 256 when an external force is applied to the actuator 256. The cap 250 can be formed of any suitable compliant material to allow the flex ring 257 to axially flex when the actuator 256 is actuated. For example, the cap 250 may be formed of polyethylene, polypropylene, copolyester, poly ether block amide, or thermal plastic elastomer. Other materials are contemplated. In some embodiments, the cap 250 is formed from two materials using a two-shot technique. A rigid material may be used for threads and a support rings and an elastomeric material may be used for a sealing surface, flex ring, or actuator.

FIG. 2D illustrates a bottom view of the cap 250 including a protrusion 254. As illustrated, the protrusion 254 extends from the actuator 256 into a medium chamber 255. The protrusion 254 includes four blades extending radially outward from a central point. In other embodiments, the protrusion 254 may include one, two, three, five, or more blades. The medium chamber 255 can be defined, in part, by the top wall 258 and a sealing flange 253. The seal member 290 (not shown) may be coupled to the sealing flange 253 to seal the medium chamber 255 using any suitable technique, such as welding, bonding, induction sealing, mechanical fitting, or gluing. Other techniques are contemplated. A liquid medium can be disposed within the medium chamber 255. A volume of the medium chamber 225 can be defined by a height H₁ and a diameter D₁ of the sealing flange 253. The height H₁ can range from about 2 millimeters to about 25 millimeters, and the diameter D₁ can range from about 10 millimeters to about 50 millimeter. The volume of the medium chamber 255 can range from about 0.1 milliliter to about 50 milliliters. Other heights, diameters, and volumes are contemplated within the scope of this disclosure. In certain embodiments, the medium chamber 255 may be partially filled.

FIG. 2E illustrates a cross-sectional view of a portion of the specimen device 200 in a ready state. As illustrated, the cap 250 is rotationally coupled to the collection member 212 of the vial 210. The flex ring 257 and the actuator 256 are in a neutral position. The protrusion 254 extends into the medium chamber 255 from the top wall 258 and does not contact the seal member 290. The seal member 290 is coupled to the sealing flange 253 to seal a liquid medium 204 within the medium chamber 255.

FIG. 2F illustrates a cross-sectional view of a portion of the specimen device 200 in an actuated state. As illustrated, an external force, represented by an arrow, is applied to the actuator 256 causing the flex ring 257 and the actuator 256 to be deflected toward the seal member 290. As the actuator 256 is deflected, the protrusion 254 can be displaced toward the seal member 290 to rupture the seal member 290. When the seal member 290 is ruptured, the liquid medium 204 contained within the medium chamber 255 can flow from the medium chamber 255, through the ruptured seal member 290, into the collection member 212, and into the vial 210 to mix with a biological specimen.

FIGS. 3A and 3B illustrate another embodiment of a specimen device 300 including a cap 350. As illustrated in FIGS. 3A and 3B, the cap 350 includes a height H₂ and a diameter D₂. The height H₂ can range from about 2 millimeters to about 25 millimeters, and the diameter D₂ can range from about 10 millimeters to about 50 millimeters. The height H₂ and the diameter D₂ defines a volume of a medium chamber 355 ranging from about 0.1 milliliters to about 50 milliliters. Other heights, diameters, and volumes are contemplated. The cap 350 includes a top wall 358, including a flex ring 357 and an actuator 356 circumferentially surrounded by the flex ring 357. A protrusion 354 extends from the actuator 356 into the medium chamber 355. A height H₃ of the protrusion 354 can facilitate rupture of a seal member 390 (not shown) when the actuator 356 is actuated. The height H₃ may range from about 2 millimeters to about 25 millimeters. In certain embodiments, the height H₃ is configured such that the protrusion 354 is contained within the medium chamber 355 without puncturing the seal member 390 until the actuator 356 is actuated.

FIG. 4 illustrates another embodiment of a specimen device 400 including a cap 450. As illustrated, the cap 450 includes a top wall 458 including a flex ring 457 and an actuator 456 circumferentially surrounded by the flex ring 457. A safety ring 459 extends upward from the top wall 458 and circumferentially surrounds the flex ring 457. A height H₄ of the safety ring 459 can range from about 1 millimeter to about 10 millimeters. The safety ring 459 may prevent inadvertent actuation of the actuator 456 by an external force causing a seal member 490 (not shown) to be inadvertently ruptured.

FIGS. 5A-5D illustrate another embodiment of a specimen device 500. As illustrated in FIG. 5A, the specimen device 500 includes a vial 510 defining a specimen chamber 515, a collection member 512, and a cap 550. The cap 550 can be coupled to the collection member 512 by a hinge 518. The hinge 518 can be a living hinge having thick portions adjacent the collection member 512 and the cap 550 and a thin portion disposed between the thick portions. The collection member 512 may include a snap ring 517 configured to sealingly engage with a snap ring 560 of the cap 550 such that the cap 550 can be snapped onto the collection member 512 without rotation. A tab 561 can extend radially outward from the cap 550 to facilitate handling of the cap 550.

The cap 550 includes an actuator 556, a seal member 590, a reinforcement member 591, a medium chamber 555, and a plunger 570 disposed within the medium chamber 555. The actuator 556 can be domed shaped to allow the actuator 556 to be deflected by an external force toward the medium chamber 555. Other shapes of the actuator 556 are contemplated within the scope of this disclosure.

FIG. 5B illustrates the plunger 570. The plunger 570 can include six blades or arms 571 extending radially outward from a central axis of the plunger 570. In other embodiments, the number of blades 571 may be two, three, four, or more. The blades 571 can diverge from a central point 574 in alignment with the central axis. The central point 574 can be sharp to puncture the seal member 590. Each of the blades 571 includes a beveled portion 572 adjacent the central point 574 and a blunt portion 573 peripheral to the beveled portion 572. The beveled portion 572 can cut through the seal member 590 following puncture of the seal member 590 by the central point 574. The blunt portion 573 can prevent the plunger 570 from cutting through the seal member 590 to retain the plunger 570 within the medium chamber 555. The plunger 570 may be formed of a rigid material, such as polycarbonate, polypropylene, polyethylene, cyclic-olefin-polymer (COP), cyclic-olefin-copolymer (COC), amorphous nylon, copolyester, acetal, polyetherimide, polyetheretherketone, acrylonitrile butadiene styrene, or a metal. Other materials are contemplated.

FIG. 5C illustrates a cross-sectional view of the cap 550 and the plunger 570 disposed within the medium chamber 555. The plunger 570 may be symmetrical about a horizontal axis. In other words, the plunger 570 may include a central point 574a that is directed towards the seal member 590 and a central point 574b that is directed towards the actuator 556. When an external force is applied to the actuator 556, the actuator 556 can interface with the central point 574b and displace the plunger 570 towards the seal member 590 causing the central point 574a to puncture the seal member 590. Beveled portions 572a of the blades 571 can cut through the seal member 590 to allow a liquid medium to flow from the medium chamber 555. Blunt portions 573a of the blades 571 can abut the seal member 590 to prevent the plunger 570 from passing through the seal member 590.

FIG. 5D illustrates an alternative embodiment of a cap 550a of the specimen device 500. As illustrated, the cap 550a includes a plunger 570a. Further, the cap 550a may include a retention ring 563 extending into a medium chamber 555a from a side wall 549a of the cap 550a. The plunger 570a can include a first detent 575 and a second detent 576 disposed at an end of a blade 571a. In a ready state, the first detent 575 may engage the retention ring 563 to prevent the plunger 570a from being displaced towards a seal member 590a. When an actuator 556a is actuated by an external force causing the actuator 556a to interface with the plunger 570a, the first detent 575 can be displaced past the retention ring 563 as the plunger 570a is displaced towards the seal member 590a by the actuator 556a. The second detent 576 may engage the retention ring 563 to retain the plunger 570a within the medium chamber 555a following rupture of the seal member 590a by the plunger 570a.

FIGS. 6A-6C illustrate another embodiment of a specimen device 600. As illustrated in FIG. 6A, the specimen device 600 includes a vial 610, a collection member 612, and a cap 650. The vial 610 can include a tubular body 611. The tubular body 611 may include external features 626 (e.g., threads) disposed adjacent a proximal end of the tubular body 611. The collection member 612 can include flexible members 619 configured to engage the external threads 626 when the collection member 612 is coupled to the vial 610. The cap 650 may be coupled to the collection member 612 via a flexible hinge 618. The cap 650 can include a seal member 690, a reinforcement member 691, and an actuator 656.

As illustrated in FIG. 6B, the cap 650 may further include a plunger 670 including a spike 680 disposed within a medium chamber 655. FIG. 6C illustrates the plunger 670. In the illustrated embodiment, the spike 680 includes a central point 674 aligned with a central axis of the plunger 670 and blades 671 extending radially outward from the central point 674. The blades 671 can include a sharp bevel edge. The central point 674 can puncture the seal member 690, and the blades 671 can cut through the seal member 690 to provide an opening in the seal member 690. The spike 680 is disposed on a first set of arms 679 extending radially outward from the central axis of the plunger 670. The arms 679 can be blunt to prevent the arms 679 from cutting through the seal member 690. A second set of arms 677 can include a domed surface configured to interface with the actuator 656. A central portion 678 may be disposed between the first set of arms 679 and the second set of arms 677 such that the first set of arms 679 and the second set of arms 677 are axially spaced apart to allow the arms 679 to flex when the plunger 670 is depressed and the arms 679 are partially supported by the seal member 690.

Referring again to FIG. 6B, the plunger 670 can be disposed within the medium chamber 655 such that the spike 680 is directed toward the seal member 690 and the dome surface of the second set of arms 677 interfaces with the actuator 656. When an external force is applied to the actuator 656, the actuator 656 interfaces with the second set of arms 677 and displaces the plunger 670 towards the seal member 690. The central point 674 can puncture the seal member 690 and the blades 671 can cut through the seal member 690 forming a hole through the seal member 690 to allow a liquid medium to flow from the medium chamber 655. The first set of arms 679 may interface with the seal member 690 to retain the plunger 670 within the medium chamber 655.

FIGS. 7A-7C illustrate another embodiment of specimen collection device 700. As illustrated in FIG. 7A, the specimen collection device 700 can include a collection member 712, a cap 750, and a tether 748. The cap 750 may be coupled to the collection member 712 by the tether 748. FIG. 7B illustrates the tether 748 coupled to the cap 750. The tether 748 may include a ring 764 and an extension 769 disposed between the ring 764 and the cap 750. In other embodiments, the extension 769 may attach the cap 750 directly to the collection member 712. FIG. 7C illustrates the collection member 712. The collection member 712 can include flanges 720 extending radially outward from the collection member 712. The flanges 720 may be disposed in two circumferential rows with a space between the rows sized to accommodate the ring 764. The flanges 720 can axially retain the ring 764 when the ring 764 is disposed around the collection member 712, as shown in FIG. 7A.

FIGS. 8A and 8B illustrate another embodiment of a specimen device 800. As illustrated in FIG. 8A, the specimen device 800 can include a vial 810, a collection member 812, and a cap 850. The collection member 812 may include a pressure flange 821 configured to engage a seal member 890 when the cap 850 is coupled to the collection member 812. FIG. 8B illustrates the cap 850. The cap 850 can include protrusions 854 extending from a top wall 858 into a medium chamber 855. Each of the protrusions 854 may include a beveled blade 847 having a point 846. The protrusions 854 may be oriented at an acute angle relative to a central axis of the cap 850 and with the points 846 radial offset from the central axis. The acute angle can range from about 10 degrees to about 75 degrees.

Referring again to FIG. 8A, when the cap 850 is rotatingly coupled to the collection member 812, the pressure flange 821 may engage the seal member 890 to displace the seal member 890 towards the protrusions 854 such that a portion of the seal member 890 may be disposed between the pressure flange 821 and the sealing flange 853. When displaced, the points 846 can puncture the seal member 890 and the blades 847 can cut the seal member 890 to form a hole in the seal member 890 to allow liquid medium to flow from the medium chamber 855. The cap 850 may be configured to rotate from about 90 degrees to about 360 degrees when coupled to the collection member 812 such that a portion of the seal member 890 is not removed and dropped into the vial 810. Said another way, the cap 850 may be configured to rotate from about a quarter turn to about a full turn.

FIGS. 9A-9C illustrate another embodiment of a specimen device 900 including a cap 950. In the illustrated embodiment of FIG. 9A-9C, the cap 950 includes a housing 992 and a seal member 990 disposed within the housing 992. The seal member 990 includes a shutter insert 993 that is fixedly coupled to the housing 992 and a rotation shutter insert 994 that is configured to rotate relative to the shutter insert 993. The shutter insert 993 can include a panel 996 and a window 995. In the illustrated embodiment, the shutter insert 993 includes four panels 996 and four windows 995 disposed between the panels 996. In other embodiments, the number of panels 996 and windows 995 may be one, two, three, five, or more. The rotation shutter insert 994 includes a panel 989 and a window 988. In the illustrated embodiment, the rotation shutter insert 994 includes four panels 989 and four windows 988 disposed between the panels 989. In other embodiments, the number of panels 989 and windows 988 may be one, two, three, five, or more. A tab 997 can extend radially outward from the rotation shutter insert 994 and through a tab opening 998 in the housing 992. The tab 997 may be utilized to rotate the rotation shutter insert 994 relative to the shutter insert 993. The size of the opening 998 and tab 997 may correspond with the size of the windows 988.

FIG. 9B illustrates the seal member 990 in a closed state where the panels 989 of the rotation shutter insert 994 may be aligned with the windows 995 of the shutter insert 993 to prevent a liquid medium from flowing from a medium chamber 955. In other words, the panels 989 of the rotation shutter insert 994 cover the windows 995 of the shutter insert 993 to selectively seal the medium chamber 955. In some embodiments, the shutter insert 993 and the rotation shutter insert 994 may be coated with a material to enhance the sealability of the seal member 990 prior to actuation. For example, the coating material can be silicone, polyurethane, or thermal plastic elastomer. Other materials are contemplated.

FIG. 9C illustrates the seal member 990 in an open state following rotation of the rotation shutter insert 994 as indicated by the arrow. In the open state, the panels 989 of the rotation shutter insert 994 can be rotationally offset from alignment with the windows 995 of the shutter insert 993 such that the liquid medium within the medium chamber 955 may flow through the windows 995.

FIG. 10 illustrates an embodiment of another specimen device 1000. In the illustrated embodiment, the specimen device 1000 may include a collection member 1012 and a spike insert 1022 disposed within the collection member 1012. The spike insert 1022 can include a spike 1023 and a support structure 1024 to hold the spike 1023 stationary relative to the collection member 1012. The spike 1023 may include a sharp point 1035. In the illustrated embodiment, the support structure 1024 can include retention feet 1025 to retain the spike insert 1022 within the collection member 1012. The retention feet 1025 can engage a distal end of the collection member 1012. In other embodiments, the spike insert 1022 may be retained within the collection member 1012 by any suitable technique, such as press fit, adhesive, welding, etc. The spike insert 1022 can be formed of a rigid material, such as polycarbonate, COP, COC, nylon, copolyester, acrylonitrile butadiene styrene (ABS), or a metal. Other materials are contemplated. In use, the sharp point 1035 may puncture a seal member of a cap when the cap is coupled to the collection member 1012 to form a hole in the seal member such that a liquid medium can flow from a medium chamber 1055 of the cap.

FIG. 11 illustrates another embodiment of another a specimen device 1100. In the illustrated embodiment, the specimen device 1100 may include a vial 1110 and a vial stand 1140. The vial stand 1140 may be configured to hold the vial 1110 in a vertical orientation. The vial stand 1140 can include a base 1141 and a vial receiver portion 1142 configured to selectively receive and hold the vial 1110 in the vertical orientation. One or more support flanges 1143 may be disposed between the base 1141 and the vial receiver portion 1142 to prevent the vial receiver portion 1142 from bending resulting in tipping of the vial 1110 and spillage of contents of the vial 1110. The base 1141 of the depicted embodiment is circular in shape. In other embodiments, the base 1141 may be of any suitable shape, such as triangular, square, pentagonal, and hexagonal. Other shapes are contemplated. The vial stand 1140 may be formed of a rigid or semi-rigid material. For example, the vial stand 1140 may be formed of polycarbonate, polyethylene, polypropylene, or metal. Other materials are contemplated.

FIG. 12A-12E illustrate another embodiment of a specimen device 1200. In the illustrated embodiment of FIG. 12A, the specimen device 1200 can include a vial 1210 defining a specimen chamber 1215 and a seal member 1290 selectively coupled to the vial 1210 to seal the specimen chamber 1215. A liquid medium 1204 may be disposed within the specimen chamber 1215. The seal member 1290 may include a tab 1236 to facilitate removal of the seal member 1290 from the vial 1210. When the seal member 1290 is removed, any suitable collection member may be coupled to the vial 1210 at the open end 1228 of the specimen chamber 1215. In one embodiment, a collection member can be rotationally coupled using external threads 1226 of the vial 1210 and internal threads of the collection member. In another embodiment, a collection member can be coupled to the vial 1210 via a press fit of a neck of the collection member into a proximal portion of the vial 1210.

Exemplary embodiments of the collection member are illustrated in FIGS. 12B-12D. FIG. 12B illustrates a collection member 1212a including an opening 1216a having a racetrack shape where a length dimension is larger than a width dimension. FIG. 12C illustrates a collection member 1212b including an opening 1216b having an elliptical shape. FIG. 12C illustrates a collection member 1212c including an opening 1216c having a circular shape. Other embodiments of a collection member having an opening of other shapes are contemplated within the scope of this disclosure.

FIGS. 13A-13E illustrate another embodiment of a specimen device 1300. As illustrated in FIG. 13A, the specimen device 1300 can include a vial 1310, a collection member 1312 disposed at a proximal end of the vial 1310, a lid 1365 coupled to the collection member 1312, a medium cartridge 1331 disposed at a distal end of the vial 1310, and a stopper 1330.

FIG. 13B illustrates a cross-sectional view of the specimen device 1300 with the stopper 1330 disposed at an open end 1328 of a specimen chamber 1315 to seal the specimen chamber 1315. The medium cartridge 1331 can be disposed at a distal end of the specimen chamber 1315 and may be selectively coupled to the vial 1310 utilizing a threaded connection. The medium cartridge 1331 may include a medium chamber 1333 to contain a liquid medium, a spike plunger 1332 slidably disposed within the medium chamber 1333, and a seal member 1390 coupled to the medium cartridge 1331 to seal the medium chamber 1333. The spike plunger 1332 may include a sharp point 1337 to puncture the seal member 1390. The medium cartridge 1331 may further include an actuator 1356 disposed at a distal end. The actuator 1356 may include a dome shape to interface with a distal end of the spike plunger 1332 having a dome shape.

FIG. 13C illustrates the specimen device 1300 in use. Following collection of a biological specimen from a patient into the collection member 1312 and specimen chamber 1315, the stopper 1330 may be disposed within the vial 1310 to seal the proximal end of the specimen chamber 1315. An external force, as indicated by the arrow, may be applied to the actuator 1356 directed towards the spike plunger 1332. The actuator 1356 can deflect and interface with the distal end of the spike plunger 1332 causing the spike plunger 1332 to be displaced towards the seal member 1390. The sharp point 1337 can penetrate the seal member 1390 forming a hole in the seal member 1390. The liquid medium within the medium chamber 1333 may flow through the hole and into the specimen chamber 1315 to mix with the collected specimen.

FIG. 13D illustrates a method of obtaining a sample of the specimen and liquid medium mixture for analysis. As illustrated, the medium cartridge 1331 can be removed from the vial 1310 and a sampling device 1302 (e.g., syringe and needle) can be inserted through a distal opening of the specimen chamber 1315 to obtain a sample of the mixture.

FIG. 13E illustrates another method of obtaining a sample of the specimen and liquid medium mixture for analysis. As illustrated, the medium cartridge 1331 may remain coupled to the vial 1310 and a sampling device 1302 (e.g., syringe and needle) can be inserted through the stopper 1330 into the specimen chamber 1315 to obtain a sample of the mixture.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified. For example, a method of compounding medicaments may include one or more of the following steps: preparing a single dose vial comprising a first liquid medicament; inserting an ampule comprising a second liquid medicament into a chamber of the single dose vial; puncturing a septum of the ampule with a needle of the single dose vial; injecting the first liquid medicament into the ampule to compound with the second liquid medicament; applying a force to a stopper of the single dose vial with the ampule; displacing the stopper toward a closed end of the single dose vial; and displacing the first liquid medicament through the needle into the ampule. Other steps are also contemplated.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment.

It will be appreciated that various features are sometimes grouped together in a single embodiment, figure, or description thereof for the purpose of streamlining the disclosure. Many of these features may be used alone and/or in combination with one another.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

The directional terms “distal” and “proximal” are given their ordinary meaning in the art. That is, the distal end of a medical device means the end of the device furthest from the practitioner during use. The proximal end refers to the opposite end, or the end nearest to the practitioner during use. As specifically applied to a specimen device of this disclosure, the proximal end of the device refers to the end nearest a collection member and the distal end refers to the opposite end.

“Fluid” is used in its broadest sense, to refer to any fluid, including both liquids and gases as well as solutions, compounds, suspensions, etc., that generally behave as fluids.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially perpendicular” is recited with respect to a feature, it is understood that in further embodiments, the feature can have a precisely perpendicular configuration.

The terms “a” and “an” can be described as one, but not limited to one. For example, although the disclosure may recite a housing having “a stopper,” the disclosure also contemplates that the housing can have two or more stoppers.

Unless otherwise stated, all ranges include both endpoints and all numbers between the endpoints.

Recitation in the claims of the term “first” with respect to a feature or element does not necessarily imply the existence of a second or additional such feature or element.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A specimen collection and transport device, comprising:
a vial comprising:
a tubular body defining a sample chamber comprising a closed distal end and an open proximal end; and
a collection member disposed adjacent the open proximal end and in fluid communication with the sample chamber; and
a cap configured to be selectively coupled to the collection member comprising:
an inner seal flange configured to sealingly engage with an inner surface of the collection member; and
a medium chamber configured to retain a liquid medium.

2. The specimen collection and transport device of claim 1,
wherein the collection member comprises a funnel shape tapering inwardly toward the specimen chamber; and
wherein the collection member is configured to receive a biological specimen.

3. The specimen collection and transport device of claim 2, wherein the biological specimen comprises any one of saliva, sputum, blood, digestive secretions, and any combination thereof.

4. The specimen collection and transport device of claim 1,
wherein the collection member comprises external threads;
wherein the cap comprises internal threads; and
wherein the external threads of the collection member are configured to threadingly engage with the internal threads of the cap when the cap is coupled to the collection member.

5. The specimen collection and transport device of claim 1,
wherein the collection member comprises a first lip extending radially outward from a proximal end of the collection member;
wherein the cap comprises a second lip extending radially inward from a distal end of the cap; and
wherein the first lip is configured to engage with the second lip when the cap is coupled to the collection member.

6. The specimen collection and transport device of claim 1, further comprising a flexible hinge disposed between the collection member and the cap.

7. The specimen collection and transport device of claim 1,
wherein the cap further comprises a tether configured to be coupled to the collection member; and
wherein the tether comprises;
a tether ring; and
a tether extension disposed between the cap and the tether ring.

8. The specimen collection and transport device claim 1, wherein the tubular body and the collection member comprise a unibody construct.

9. The specimen collection and transport device of claim 1, wherein the tubular body and the collection member are separate components, and wherein the collection member is configured to be selectively coupled to the tubular body.

10. The specimen collection and transport device of claim 1, wherein the liquid medium comprises any one of a preservative, an antibiotic, a pH indicator, a buffering agent, and any combination thereof.

11. The specimen collection and transport device of claim 1, wherein the cap further comprises:
an actuator and a flexible ring circumferentially surrounding the actuator;
a protrusion extending from the actuator into the medium chamber; and
a seal member selectively coupled to the seal flange.

12. The specimen collection and transport device of claim 11,
wherein the actuator is configured to be actuated in response to an external force directed toward the medium chamber;
wherein the protrusion is configured to be displaced by the actuator toward the seal member to rupture the seal member;
wherein the liquid medium is configured to flow from the medium chamber, through the ruptured seal member, and into the sample chamber; and
wherein the liquid medium is configured to mix with a biological sample within the sample chamber.

13. The specimen collection and transport device of claim 11,
wherein the protrusion comprises a plurality of blades extending radially outward from a central point; and
wherein a number of the plurality of blades ranges from one to eight.

14. The specimen collection and transport device of claim 11, wherein the seal member comprises a metal foil.

15. The specimen collection and transport device of claim 11, further comprising a reinforcement member selectively coupled to the seal member.

16. The specimen collection and transport device of claim 11,
wherein the cap further comprises a safety ring disposed circumferentially around the flexible ring and extending upward from the cap; and
wherein the safety ring is configured to prevent inadvertent actuation of the actuator.

17. A method of collecting and transporting a biological specimen, comprising:
collecting the biological specimen in a vial;
coupling a cap to an open end of the vial;
actuating the cap to rupture a seal member;
releasing a liquid medium from a media chamber; and
mixing the liquid medium with the biological specimen.

18. The method of claim 19, wherein actuating the cap comprises:
depressing an actuator of the cap toward the seal member;
displacing a protrusion extending from the actuator, wherein the protrusion ruptures the seal member.

19. The method of claim 19, wherein the biological specimen comprises any one of saliva, sputum, blood, digestive secretions, and any combination thereof.

20. The method of claim 19, wherein the liquid medium comprises any one of a preservative, an antibiotic, a pH indicator, a buffering agent, and any combination thereof.
